(19) United States
(12) Patent Application Publication
(10) Pub. No.: US 2010/0261212 A1
(43) Pub. Date: Oct. 14, 2010

(54) KINETICS OF MOLECULAR RECOGNITION MEDIATED NANOPARTICLE SELF-ASSEMBLY

(76) Inventors: Chinmay Prakash Soman, Nashville, TN (US); Todd Donald Giorgio, Nashville, TN (US)

Correspondence Address: STITES & HARBISON PLLC, 401 COMMERCE STREET, SUITE 800, NASHVILLE, TN, 37219, US

(21) Appl. No.: 12/685,597

(22) Filed: Jan. 11, 2010

Related U.S. Application Data
(60) Provisional application No. 61/143,659, filed on Jan. 9, 2009.

(51) Int. Cl. G01N 33/53; C12Q 1/42

(52) U.S. Cl. 435/21; 436/501; 977/774; 977/920

(57) ABSTRACT

A method of multiple protein biomarker detection, comprising providing at quantum dot-antibody conjugates that have an affinity for at least two different protein biomarkers; contacting the conjugates with a sample from a subject; allowing the proteins to bridge the antibodies, forming protein biomarker/quantum dot-antibody conjugate agglomerates; detecting the presence of the biomarkers by excitation of the agglomerates.

### PRIOR APPLICATION INFORMATION

This application claims benefit to U.S. Patent Application No. 61/143,659, filed Jan. 9, 2009. The contents of said application are incorporated herein by reference in their entirety.

### FIELD OF THE INVENTION

The present invention generally relates to the field of biomarkers, and molecular recognition elements.

One embodiment of the present invention is a method for the detection of multiple biomarkers.

Another embodiment of the present invention is a method for the detection of cancer, including breast cancer.

Another embodiment of the present invention is a device used in the detection of biomarkers and molecular recognition elements.

Another embodiment is the use of detectable particles that can be coupled to a molecular recognition molecule that specifically binds to a biomarker of interest, and can form detectable agglomerates through self-assembly.

In embodiments, the detectable particle can be a quantum dot, nanotube, nanoparticle, nanofiber, etc.

Yet another embodiment of the present invention is the use of quantum dot agglomerates for assessment of cancer or other disease.

In aspects of the present invention, molecular recognition mediated self assembly of quantum dot-antibody conjugates exhibits a concentration dependant sigmoidal kinetic behavior. The parameters of the kinetic behavior may be utilized for quantification of proteomic biomarkers in solution by characterizing non-steady state aggregate population.

Nanoscale quantum dot-antibody conjugates have been shown to self-assemble to form micron-scale aggregates in the presence of specific proteomic antigen. The self-assembly process exhibits sigmoidal kinetics, suggesting that nucleation limits aggregation. Self-assembly kinetics in this study are characterized by flow cytometric analysis of the aggregation reaction over time. A range of physiologically relevant concentrations of the protein angiopoietin-2, a candidate cancer biomarker, are incubated with quantum dots conjugated with a polyclonal mixture of anti-angiopoietin-2 antibodies. Antigen concentration modulates the slopes and inflection times of the sigmoidal kinetics curves. The self-assembly kinetics embodiments of the present invention allows for improvements in sensitivity and specificity of this novel proteomic biomarker detection technique and improve the screening, diagnostics, and therapy response monitoring for cancers and other diseases.

### BACKGROUND OF THE INVENTION

Early detection of cancer has been demonstrated to significantly improve clinical outcomes. Advances in cancer biology and technology support increasingly important, emerging efforts to develop methods for early detection of metastatic disease and for the rapid assessment of individual response to cancer treatment. For example, the five year survival of patients with metastatic breast cancer is only 23%, representing a decreased survival due to metastasis of 74 patients out of every 100 diagnoses of distributed disease. Variability in patient response to treatment is currently regarded as another critical factor in controlling breast cancer outcomes, but the current clinical methods available for such assessments are slow, expensive and inaccurate.

The three most common primary cancers—lung, breast, and prostate—often metastasize to bone. Bone metastases are one of the most frequent causes of pain in people with cancer, and cause complications that have significant adverse effect on health and lifestyle. Proteomics offers a novel and powerful way of alleviating these problems by diagnosing bone metastases early. The diagnostic and prognostic power of proteomic biomarkers is enhanced by simultaneous interrogation of multiple biomarkers, but the sophisticated, research oriented analytical tools used for the discovery of protein biomarkers such as mass spectrometry, gel electrophoresis and protein arrays are not suitable for frequent and low-cost ‘point-of-care’ testing. The use of multiple traditional ELISA assessments, one for each biomarker, is possible, but broad adoption of this approach requires expensive, centralized robotic instrumentation that is not widely available. A new method for the detection of multiple biomarkers is urgently needed to enable the translation of emerging proteomic results to routine clinical use in assessing bone metastasis status.

The present invention helps meet these needs. Embodiments of the invention allow for low-cost, minimally invasive approach with high sensitivity and specificity for detecting bone metastasis status—especially one that can be conducted rapidly and conveniently in a physician's office—to enable early detection of cancer stages, a critical unmet need in the challenge to eradicate deaths due to cancer. Additional embodiments include the use of the same device to detect alternate biomarkers associated with response to therapy, which reduces patient exposure to ineffective therapies, minimize the development of drug-resistant disease and improve outcomes through rapid identification of treatment options with the greatest efficacy for a particular patient. An extension of the present invention includes monitoring disease relapse following successful treatment in a sensitive and rapid way at the ‘point-of-care’, effectively addressing a critical concern shared by all cancer survivors.

### BRIEF SUMMARY OF THE INVENTION

Aspects of the present invention include novel approaches to sensitive and rapid antigen detection. In embodiments of the present invention, in the presence of a specific biomarker, detectable particle-molecular recognition element conjugates rapidly self-assemble into agglomerates that are typically more than one order of magnitude larger than their individual components. The size distribution of the agglomerated colloids depends on, among other things, the relative concentration of quantum dot conjugates and antigen molecules. These agglomerates, mediated by antigen recognition, are, in embodiments of the invention, characterized by measuring their light scattering and fluorescence characteristics in an unmodified flow cytometer. Protein antigens angiopoietin-2 and mouse IgG are two examples that can be detected to sub picomolar concentrations using this embodiment.

The present invention provides relatively simple techniques to enable the potential simultaneous detection of multiple antigenic biomarkers directly from physiological media and could be used for early detection and frequent screening of cancers and other diseases.

One embodiment of the present invention is a method of detecting a biomarker that comprises (a) coupling detectable particles to a molecular recognition element that binds to a biomarker of interest to form functionalized conjugates; (b) providing a sample from a subject; (c) introducing the sample and the functionalized conjugates to at least one sample holding channel in an incubation chamber to form agglomerates through self-assembly if contacted with the corresponding biomarker; and (d) detecting the agglomerate by excitation to determine the presence of the biomarker.

Another embodiment is a method of multiple protein biomarker detection that comprises (a) providing at least two detectable particle-molecular recognition element conjugates that have an affinity for at least two different protein biomarkers; (b) contacting the conjugates with a sample from a subject; (c) allowing the proteins to bridge the molecular recognition, forming detectable agglomerates; and (d) detecting the presence of the biomarkers by excitation of the agglomerates.

Another embodiment is a method of monitoring a response to a treatment therapy that comprises (a) providing a detectable particle-molecular recognition element conjugate that has a selective affinity for a biomarker; (b) providing a sample from a patient; (c) introducing the conjugate to the sample, forming detectable conjugate/biomarker agglomerates through self-assembly; (d) detecting the agglomerate to obtain a first quantitative result; (e) after a passage of time, providing a second sample from the patient; (f) introducing the second conjugate to the sample, forming detectable conjugate/biomarker agglomerates through self-assembly; (g) detecting the resulting agglomerate to obtain a second quantitative result; and (h) comparing the first and second quantitative result.

As shown below in Example 8, directed self-assembly of nanostructures into microstructures through intermolecular interactions is an important phenomenon in many biological systems. Assembly of virus coat proteins into capsids, of microtubulin into microtubules, and of collagen and fibrinogen into their respective fibrils are just a few examples where self-assembly plays a critical role in biological processes. Programmed self assembly using biomolecular interactions as a route to synthesis of novel nanostructured materials has also been an area of active investigation in the recent past. Techniques aimed at molecular diagnostics have also been demonstrated using nanoparticle self assembly mediated by molecular interactions, including polynucleotide interactions and antibody-antigen interactions.

The present inventors have demonstrated a novel technique for sub-picomolar quantitative detection of proteomic antigens using single step fluid phase incubation. In this technique, quantum dots (QD) conjugated with polyclonal antibodies (Ab) through the streptavidin-biotin interaction are incubated with specific antigens in a physiological buffer. The molecular recognition between the antibodies and antigens causes aggregation of the nanoscale conjugates and proteins, resulting in the formation of microscale structures. These larger structures can be distinguished from the individual components based on differences in light scattering properties and by other analytical techniques. By sequentially characterizing very small volumes of the reaction mixture, such as via flow cytometry, the microscale aggregates can be quantified. At equilibrium, the antigen concentration and the fraction of events that are classified as aggregates are correlated through a log-linear relationship. In another embodiment, multiple quantum dot populations with distinct fluorescence emissions are used for multiplexed detection of two antigens. This embodiment can be extended through the use of alternately biofunctionalized QD with additional fluorescence emission characteristics.

An embodiment of the present invention is the kinetics of QD-Ab aggregation mediated by angiopoietin-2 is described. The kinetics of self assembly of bio-macromolecules has been studied for many systems, including virus capsid assembly, microtubule formation, fibril assembly and other protein aggregation phenomena. Theoretical and computational studies on these systems illuminate the mechanisms that drive the self-assembly processes. Previous work has also examined factors that influence the kinetics, such as the concentration of various moieties, agitation and presence of agents that promote or inhibit intermolecular interaction. These factors may be utilized for promotion or inhibition of aggregation. Understanding the kinetics of the present system improves the specificity and sensitivity of the proposed diagnostic method. Similar studies yielded improvements to the conventional surface based enzyme linked immunosorbent assay (ELISA), and informed the development of the kinetic ELISA method. While the current study explores the kinetics of highly specific molecular interaction mediated self-assembly, nanoscale material aggregation is important in a wide variety of applications. Controlled aggregation and/or prevention of non-specific aggregation are important considerations for various technologies that use nanomaterials, regardless of the presence or absence of molecular recognition. Thus, the techniques of this embodiment of the present invention provide novel methods of quantitatively characterizing important aspects of nanoscale phenomena.

### BRIEF DESCRIPTION OF THE DRAWINGS

FIG. 1 is an illustration showing surface functionalized Quantum Dots to promote self-assembly in the presence of biomarker proteins specifically associated with breast cancer status. Aggregates may be optically analyzed in a hand-held microfluidic device to determine if the self-assembled structures match the target biomarker profile and provide a rapid, inexpensive assessment of breast cancer status at the ‘point-of-care’.

FIG. 2 shows an example of a micropatterened fluidic chip of the present invention (left) and an example of a hand-held electronic device of the present invention (right). The chip of the present invention, typically a disposable, inexpensive microfluidic chip can be designed to provide the blood sample handling, QuaD-MAP reagent mixing and spatial separation of the resulting self-assembled QD aggregates. It can simply be inserted into a hand-held electronic device that optically interrogates the QD aggregates and interprets the fluorescence patterns to create an assessment of breast cancer status.

FIG. 3 is a graph showing nanoscale QDs surface functionalized with GaM antibody self-assemble into microscale aggregates mediated by polyvalent antigen (red peak at 2,000 nm). Addition of nonspecific antigen (Hum) fails to mediate large aggregate formation (lack of grey peak >700 nm).

FIG. 4 is a set of graphs that show, though flow cytometry, detection of antigen, achieved by characterizing agglomerates as a function of total events. This figure shows self-assembly of QD-GaM-IgG aggregates modulated by antigen concentration (A through C reflect [Mus] of 0, 1, 100 μg/mL, respectively. 10 μg/mL not shown). 100 μg/mL of control antigen (Hum, D) mediates the self-assembly of smaller and fewer aggregates than Mus. Regions R1 R2 R3 and R4 correspond to Calibration particles with mean diameters of 200, 1000, 2000 and 2866 nm respectively.

FIG. 5 is a schematic of electrokinetically controlled flow focusing in a 60 μm wide cross microchannel (left). The left end of the horizontal channel is a sample reservoir filled with a blood solution containing the to-be-detected particles and the right end is a waste collection reservoir. The ends of the vertical channel are each connected to reservoirs filled with a buffer solution. One electrode is inserted in each of the four reservoirs. Voltages applied to the electrodes generate electroosmotic flows in the microchannel. These voltages can be adjusted to control the fluid flow rates so that the two side flows (buffer solution) will squeeze the central particle-carrying flow to a desired size, and hence realize the stream focusing and particle separation functions. The electroosmotic flows in the microchannel are laminar flows and don't mix between streams. An example of the focused fluorescent particle stream (entering the cross intersection from left, and becoming a line of single particles after the intersection) (left). The arrows indicate the flow directions.

FIG. 6 shows optical fibers are embedded in a PDMS chip for particle detection (left). The thinner fiber introduces the laser emission and the thicker fiber couples to the optical detector. A particle is detected as it passes through the laser beam. The detected optical signal strength is shown (right) where each peak represents one particle.

FIG. 7 is an illustration of an example of a microfluidic chip of the present invention and an optical detection system of the present invention. Other embodiments can include additional, parallel optical detection subassemblies to enable light scattering intensity and four wavelength fluoresce intensity measurements on each particle and refined blood sample handling with integrated QD/plasma mixing.

FIG. 8 is graph showing agglomeration behavior including agglomeration percentage and amount of biomarker present in the sample.

FIG. 9 is multiple graphs showing flow cytometric detection of a biomarker achieved by characterizing agglomerates as a fraction of total events.

FIG. 10 shows aggregation kinetics by flow cytometric analysis of ang2 mediated QD-aA2 aggregation at specific time points. Aggregates are quantified as a fraction of total events. The panels a, b and c show the raw flow cytometric data depicting increasing aggregate formation with time. This data was acquired for the samples with 10 pM QD-aA2 and 10 pM ang2 at 5, 30 and 45 minutes in panels a, b and c, respectively. Each dot represents one particle or ‘event’ detected. Forward light scatter (FSC-H) and side light scatter (SSC-H) intensities are positively correlated with the size and complexity of the particles. Optimization of the detection parameters enables the resolution of micron scale aggregates (red dots, upper right quadrant) from the individual QD-aA2 conjugates and small aggregates (black dots, bottom left quadrant). The ovals labeled R1, R2, R3, R4 and R7 indicate 0.2, 0.5, 1, 2 and 2.8 μm calibration particles respectively.

FIG. 11 shows the QD-aA2:ang2 system exhibiting a sigmoidal self-assembly kinetics. The parameters of aggregation kinetics in this system are affected by ang2 concentration, as detailed in Table 1. With increasing ang2 concentration, the aggregation rate increases, and time to inflection point (t₀) and time to reach steady state aggregate fraction decreases. The difference between the slopes, including at t₀ and at t=5 minutes for the three sigmoid curves suggest the possibility of antigen detection and quantification based on rate of increase in aggregate percent, rather than based on the equilibrium aggregate concentration as described previously. This ability may also be beneficial for detection of molecular biomarkers in complex physiological media, where non-specific intermolecular interactions may have significant effect on the aggregate fraction, especially over long incubation periods. The aggregate fraction is the fraction of total events that are in the upper right quadrant of the SSC vs. FSC dot plot (FIG. 10). Each data point in the graph represents the mean and standard deviation of data from three different experiments.

FIG. 12 shows the sigmoidal kinetics observed for QD-aA2:ang2 self assembly, showing a nucleation limited process, which is schematically depicted here. i—The entropic cost of particle aggregation may outweigh the reduction in free energy, resulting in slow nucleation. ii—The presence of multiple binding sites on nuclei of critical size leads to rapid growth of aggregates. iii—The depletion in available free ang2 causes the asymptotic approach to equilibrium aggregate fraction.

### DESCRIPTION OF EMBODIMENTS OF THE INVENTION

Embodiments of the present invention offer a novel way of fulfilling the urgent needs discussed above. The prognostic power of proteomic biomarkers is enhanced by simultaneous interrogation of multiple biomarkers. As indicated above, in the past, sophisticated, research oriented analytical tools used for the discovery of protein biomarkers such as mass spectrometry, gel electrophoresis and protein arrays are typically not suitable for frequent and low-cost ‘point-of-care’ testing.

Thus, a method for the detection of multiple biomarkers without the above limitations is needed to enable the translation of emerging proteomic results to routine clinical use in assessing cancer status, including breast cancer.

As used herein, the term “biomarker” refers to a biochemical in the body that has a particular molecular trait to make it useful for diagnosing a condition, disorder, or disease and for measuring or indicating the effects or progress of a condition, disorder, or disease. Examples of biomarkers that can be used in connection with the present invention include those suitable for agglomeration-based detection, or biomarkers that allow multivalent molecular recognition interactions. Thus, biomarkers of the present invention include proteins, protein fragments, DNA, RNA, oligosaccharides, etc.

Also, as used herein, “detectable particle” includes all units that are detectable by, for example, magnetic, color, absorption, etc. means. The detectable particles of the present invention are also capable of receiving a molecular recognition molecule that specifically binds to a biomarker of interest. The molecular recognition molecule can be a polyclonal antibody for different sentinel proteins.

The detectable particles can, of course, be nanoparticles, including quantum dots.

The molecular recognition molecule may be an antibody, including polyclonal antibodies that have an affinity for a specific biomarker.

One aspect of the present invention is the design, fabrication and assessment of a new approach that will provide breast cancer patients with a sensitive, minimally invasive and near-real-time assessment of their disease status. Physicians will use this information to optimize treatment approaches on an individual basis to improve clinical outcomes and minimize discomfort.

An instrument of the present invention optimizes at least one nanoscale proteomic biomarker assay and the microfluidic device characteristics.

The methods of present invention are effective in detecting and/or quantifying various types of cancers, including but not limited to: pancreatic cancer, renal cell cancer, Kaposi's sarcoma, chronic leukemia (preferably chronic myelogenous leukemia), chronic lymphocytic leukemia, breast cancer, sarcoma, ovarian carcinoma, rectal cancer, throat cancer, melanoma, colon cancer, bladder cancer, lymphoma, mesothelioma, mastocytoma, lung cancer, liver cancer, mammary adenocarcinoma, pharyngeal squamous cell carcinoma, gastrointestinal cancer, stomach cancer, myeloma, prostate cancer, B-cell malignancies or metastatic cancers.

Additionally, the microfluidic device of the present invention can be modified to detect molecular biomarkers related to different diseases including those related to neurodegenerative diseases, cardiovascular diseases, inflammation, etc.

One embodiment of the preset invention is related to the quantifiable and reproducible self-assembly in the QD-aA2:ang2 system exhibiting a sigmoidal kinetic behavior, comparable to that observed in other self-assembly processes based intermolecular interaction. The kinetics of aggregation shows that the rate of aggregation and the equilibrium extent of aggregation are sensitive to the antigen concentration. Due to its unique characteristics, this multifunctional nano-conjugate based analyte detection embodiment provides a novel, simple, and rapid mechanism for detecting molecular biomarker from physiological samples. An understanding of the aggregation kinetics and mechanisms will be particularly important in developing practical implementations of this technology, especially for complex samples containing non specific antigens with weak intermolecular interactions that may support weak QD-Ab aggregation. Nanoscale particle aggregation in solutions is a fundamental phenomenon that affects various technologies that include nanomaterials.

As indicated above, another aspect of the present invention is the use of QuaD-MAP for assessment of bone metastasis status in a hand-held microfluidic device, as described in PCT/US2008/059293, incorporated herein by reference. One embodiment includes the use of at least one of TGF-β1, osteoprotegerin, parathyroid hormone related protein, and bone specific alkaline phosphatase as potential bone metastasis biomarkers. These and other biomarkers can be associated with a unique quantum dot fluorescence emission wavelength (‘color’), in a multiplexed manner. The size distribution and fluorescence characteristics of the self-assembled microscale agglomerates formed from nanoscale quantum dots (QDs) can be assessed as a function of time in conventional instrumentation with QD:antibody ratio, QD:biomarker ratio and mixing intensity as modulated parameters.

The Quantum Dot Enabled Multiplexed Antigen Profiling (QuaD-MAP) system is based on the ability of nanoparticles, quantum dots, to self-assemble—form structures without external prodding. This embodiment starts with nanoscale fluorescent beads called quantum dots. These come in a range of different colors and are used to tag specific biological structures. Another component is antibodies, proteins produced by the body's immune system that recognize and bind to foreign substances. The researchers chemically attach antibodies onto the surface of the quantum dots that bind to a particular biomarker. When they mix them in liquid containing the biomarkers, the proteins act as bridges between the quantum dots, forming microscale ‘snowballs’ from the nanoscale ‘snowflakes.’ Typically, within a matter of minutes, the fluorescent snowballs grow large enough that they can be easily detected by a flow cytometer, a standard hospital instrument used for counting and measuring blood cells. If the targeted biomarkers are not present, the quantum dots do not agglomerate and remain undetectable by the cytometer. The QuaD-MAP approach can detect the presence of a number of different biomarkers simultaneously by attaching the antibodies to each biomarker to different-colored quantum dots.

Herein the term “quantum dots” refers to semiconductor nanocrystals that can be excited with a single wavelength of light resulting in detectable fluorescence emissions of high quantum yield, and with discrete fluorescence peaks (e.g. having a narrow spectral band such as between about 10 nm to about 60 nm). Quantum dots can comprise an optical core that is surrounded by a protective shell. By way of example, the optical core can comprise a cadnium/selenium core and/or a cadium/selenium/telluride core.

The protective shell can comprise a zinc sulfide shell. The protective shell can be modified to promote water solubility and to enhance to enhance stability under complex conditions of aqueous environments encountered in living tissues and in protocols for labeling tissues. For example, the protective shell can be conjugated to methoxy terminated polyethylene glycol amine to improve the circulation half-life of the quantum dot and mitigate non-specific uptake by the reticuloendothelial system (RES). Quantum dots comprising a cadnium/selenium core, a zinc sulfide protective, and a conjugated 500 Dalton amino polyethylene glycol polymer are commercially available from Quantum Dot Corporation, Hayward, Calif. These quantum dots are capable emitting light at a wavelength of about 705 nm (.+−.about 20 nm) upon excitation of about with light having a wavelength of about 400 nm ((.+−.about 40 nm).

Examples of quantum dots that may be used in accordance with the invention can also be substantially non-toxic to living tissue (e.g., such that they can be used to label living tissue or cell processes active in living tissue) as well as be sensitive in terms of being detected in fluorescence imaging of tissue because of their fluorescence properties (e.g., including, but not limited to, high quantum efficiency, resistance to photo-bleaching, and stability in complex aqueous environments). Of course, the quantum dots are linkable to other agents, detectable particles, that include chemical compounds, such as proteins, peptides, nucleic acids, and antibodies (e.g., monoclonal and polyclonal antibodies) by conjugation to the protective shell.

Examples of quantum dots of the present invention have cores having mean diameters of less than about 20 nm, more preferably less than about 15 nm and most preferably between about 2 and about 5 nm. Mean diameters of the quantum dots can be measured using techniques well known in the art such as transmission electron microscopy. A property of quantum dots is that they emit fluorescence following exposure to exciting radiation, most usually ultraviolet light. This effect arises because quantum dots confine electrons, holes, or electron-hole pairs or so-called excitons to zero dimensions to a region on the order of the electrons' de Broglie wavelength. This confinement leads to discrete quantized energy levels and to the quantization of charge in units of the elementary electric charge. Quantum dots are particularly significant for optical applications due to their theoretically high quantum yield. Thus, compared to the conventional use of fluorescent labels that need to be continuously excited to produce fluorescence and therefore require complicated or expensive equipment for excitation and detection, the long active fluorescence lifespan produced from quantum dots is advantageous for applications in which they are used as labels. Thus, the energy levels of small quantum dots can be probed by optical spectroscopy techniques.

In addition, quantum dots have the further advantage that their energy levels, and hence the frequency of the radiation they emit, can be controlled by changing features such as the material from which the quantum dot is made, the size of the quantum dot and the shape of the quantum dot. Generally, quantum dots emit light in visible wavelengths that can be seen by the unaided eye. While the material from which the quantum dot is formed has an effect on the wavelength of the light it emits, the size of the quantum dot usually has a more significant effect on the wavelength of light it emits and hence its visible coloration. In general, the larger quantum dots emit light towards the red end of the spectrum, while smaller quantum dots emit light towards the blue end of the spectrum. This effect arises as larger quantum dots have energy levels that are more closely spaced. This allows the quantum dot to absorb photons containing less energy, i.e. those closer to the red end of the spectrum.

Additionally, as indicated above, an aspect of the invention includes microfluidic devices specifically tuned for ‘point-of-care’ assessments of metastasis status using the QuaD-MAP assay.

Another object of the present invention is the simultaneous self-assembly of quantum dots surface functionalized for the capture of biomarkers associated with breast cancer status. c-ErB-2, sEGFR, galectin-3, are examples of breast cancer biomarkers usable with the present invention. A QuaD-MAP method of the present invention is the detection of c-ErB-2 alone based on the sample processing and detection capabilities of the microfluidic device. The size distribution and fluorescence characteristics of the self-assembled microscale aggregates formed from nanoscale quantum dots will be assessed as a function of time in conventional instrumentation with QD:antibody ratio, QD:biomarker ratio and mixing intensity as modulated parameters.

Another embodiment of the present invention is an optical detection system that includes discrete fluorescence sensors for four different emission wavelengths and an additional sensor for forward light scatter intensity. Additionally, this embodiment may implement a feedback loop that controls electrokinetic particle separation by modulation of reservoir voltage potentials under the control of real-time optical detection information.

Microfluidics is the science and technology of fluid flow and mass (molecules, particles and cells) transport in microscale channels. The ‘lab-on-a-chip’ approach is a miniaturized biomedical laboratory built on a glass or plastic chip with a size of several centimeters on each side. Typically, such a chip has microchannels, wells and built-in sensors. External pressure or electric potential is applied to transport liquids and particles in the microchannels. ‘Lab-on-a-chip’ devices can perform various biomedical tests and diagnoses (such as detecting viruses or bacteria), replacing conventional, room-based biomedical laboratories. The key advantages of these ‘lab-on-a-chip’ devices include dramatically reduced sample/reagent consumption, very short analysis time, high throughput, automation and portability. Embodiments of the present invention develop new microfluidic ‘lab-on-a-chip’ capabilities, integrated with the QuaD-MAP assay, to synergistically provide a new approach for sensitive breast cancer assessment at the ‘point-of-care’.

When a solid surface is in contact with an aqueous solution, electrostatic charge will be established at the surface. These surface charges attract the counter ions close to the solid-liquid interface, forming an electrical double layer (EDL). Such an EDL field is responsible for two basic electrokinetic phenomena: electroosmosis and electrophoresis. When an external electrical potential is applied tangentially to the solid surface, the excess counter ions in the EDL will move under the influence of the applied electrical field, pulling the liquid with them and resulting in electroosmotic flow. The liquid movement is carried through the rest of the liquid in the microchannel by viscous effects. Electroosmotic flow can generate the required flow rate in very small microchannels without an applied pressure difference cross the channel. Additionally, using electroosmotic flow to transport liquids in complicated microchannel networks does not require an external mechanical pump or moving parts and it can be easily realized by controlling the applied electrical fields via electrodes. If the surface charge of particles suspended in the fluid is not strong, or the ionic concentration of the liquid (e.g., typical buffer solutions) is high, the particle will move with the liquid. Using electrical fields to manipulate and transport particles and biological cells in microchannels is particularly suitable for ‘lab-on-a-chip’ applications. Another embodiment of the present invention relates to the integration of the components for automated blood sample preparation and QuaD-MAP processing into the disposable microfluidic chip.

Studies by the present inventors have shown self-assembled, microscale aggregate formation from biofunctionalized nanoscale QDs mediated by specific, multivalent bridging antigens.

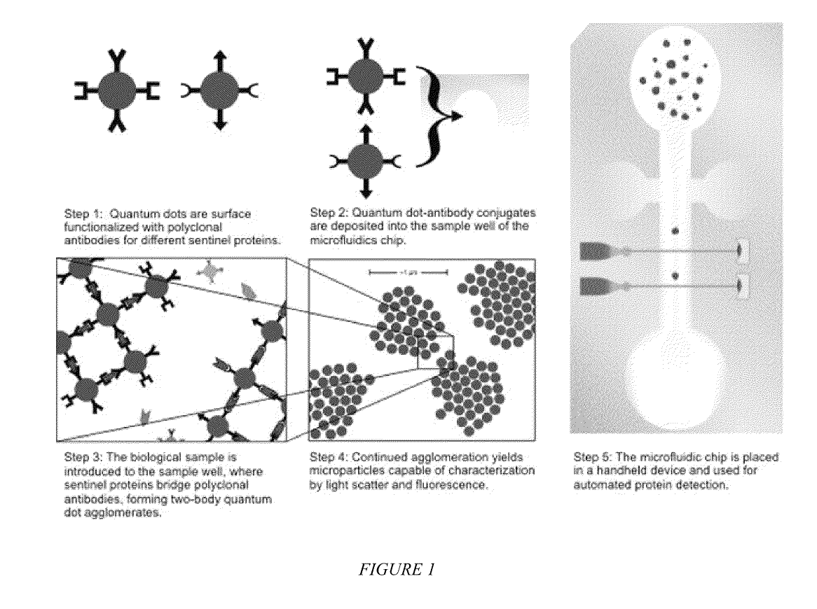

Quantum Dot enabled Multiplexed Antigen Profiling (QuaD-MAP) translates the powerful, well-known characteristics of immunoassay methods to the surface of nanostructures to create a new approach for proteomic profiling from physiological fluids such as blood, saliva or nipple aspirate (see FIG. 1). QuaD-MAP is typically based on the creation of microscale aggregates via self-assembly of nanoparticles mediated by specific biomarkers. The result is a soluble assay that can be multiplexed for simultaneous detection of many biomarkers in a minimally invasive, automated, rapid and low cost manner.

QuaD-MAP assay performance is greatly influenced by three nanoscale phenomena:

self-assembly, leading to a 1000-fold or greater change in volume (from single quantum dot (QD) nanoparticles with a volume of 1.25×10-22 m3 to micron-sized aggregates with volumes up to 1.25×10-19 m3), creating a strong differential light scattering signal in the presence of the biomarker;

high intensity fluorescence emission of QDs that enables high signal to noise ratio in biomarker detection; and

the flow-based interrogation of individual aggregates in small sample volumes that enables new assay interpretation techniques compared to conventional immunoassays that investigate bulk properties.

Together, these effects have enabled embodiments of the present invention: detection of femtomolar concentrations of a model proteins (such as mouse IgG and angiopoieten 2) using conventional clinical flow cytometers.

Embodiments of the present invention are believed to exceed the sensitivity of conventional clinical immunoassays such as ELISA and assess breast cancer status with unparalleled sensitivity. The present invention will enable QuaD-MAP assessment of breast cancer status at the ‘point-of-care’ (in a physician's office, for example) to provide rapid feedback to the patient on disease status, treatment response or relapse following successful therapy.

Thus, embodiments of the present invention relate to a low-cost, minimally invasive approach with high sensitivity and specificity for detecting cancer status, including breast cancer status—especially one that can be conducted rapidly and conveniently in a physician's office—would enable early detection of metastases, a critical unmet need in the challenge to eradicate deaths due to breast cancer. Additionally, embodiments of the present invention may be used to detect alternate biomarkers associated with response to breast cancer therapy, would reduce patient exposure to ineffective therapies, minimize the development of drug-resistant disease and improve outcomes through rapid identification of treatment options with the greatest efficacy for a particular patient. An extension of this approach includes monitoring disease relapse following successful treatment in a sensitive and rapid way at the ‘point-of-care’, effectively addressing a critical concern shared by all breast cancer survivors. The present invention allows for these approaches for breast cancer detection and status monitoring through the selection of appropriate biomarkers.

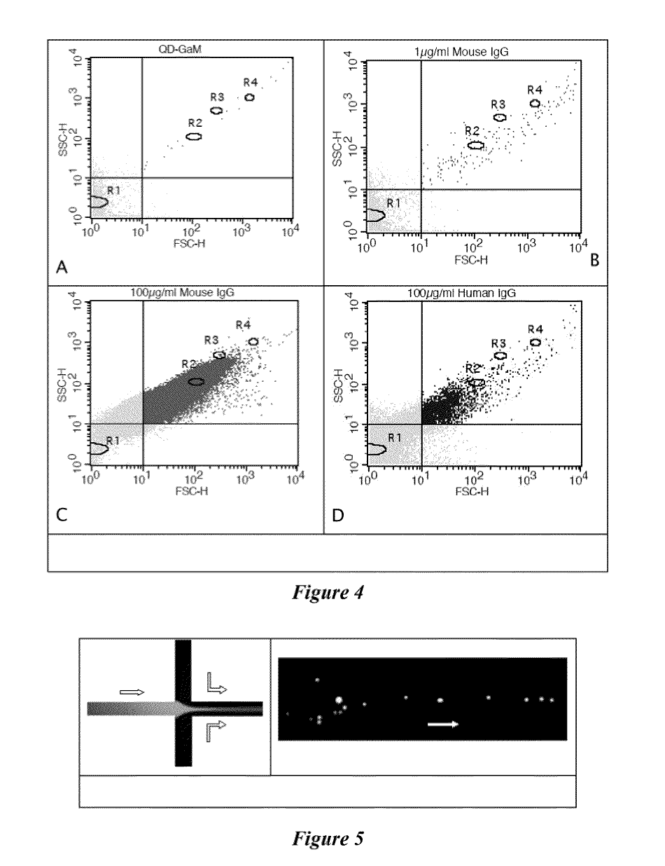

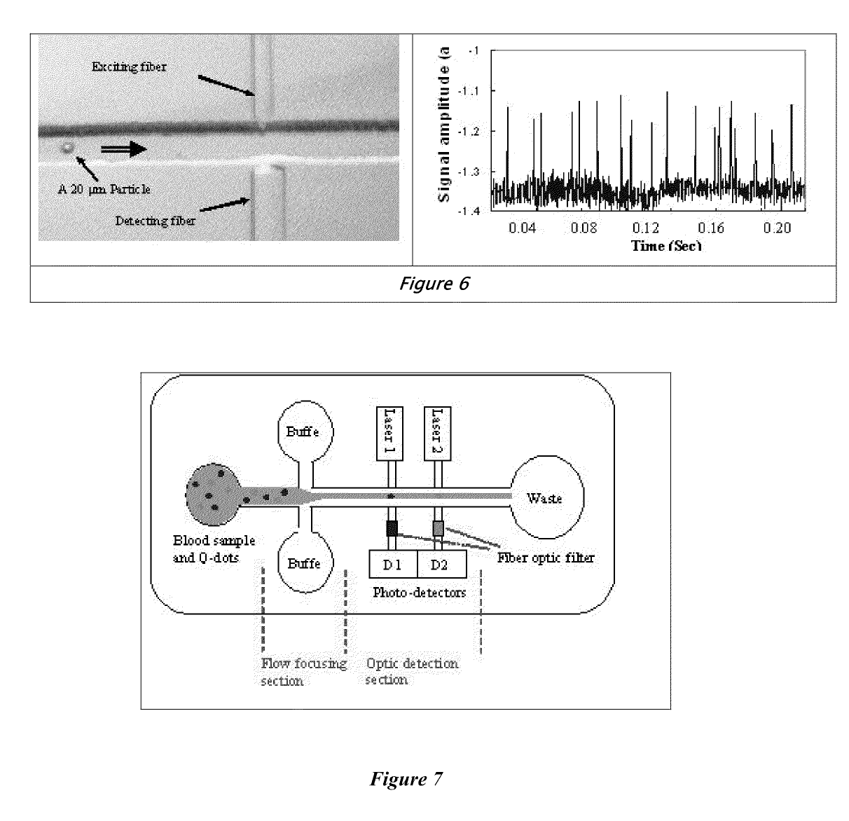

Additionally, studies have shown antigen mediated self-assembly of microscale aggregates from nanoscale particles measured by light scattering (FIG. 3) and flow cytometry (FIG. 4); antigen specificity and sensitivity in mediating self-assembly (FIG. 4); electrokinetic flow focusing of liquid and particles in microchannels (FIG. 5); and particle tracking and fluorescence characterization during electrokinetic flow in a microchannel (FIG. 6).

As stated above, an embodiment of the present invention is a portable, electrokinetic-based microfluidic chip device to characterize QD aggregates self-assembled under the influence of breast cancer biomarkers in a drop of blood. This device detects the presence of one or more breast cancer markers through enumeration of QD aggregates possessing the fluorescence emission wavelength corresponding to a specific biomarker antibody. In one embodiment, of up to four breast cancer biomarkers are detected.

Embodiments of the present invention are advantageous because of the absence of an external pump, tubing and valves and/or bulky optical detection instruments.

Embodiments can be constructed using small diode lasers, Si-PIN detectors and optical fibers. The microfluidic chip may be made by using PDMS and glass plates by a soft lithography technique and will be inserted into the reusable detection platform to conduct the assay. This design eliminates embedded waveguides or optical fibers from the chip (see FIG. 7, for example).

In embodiments of the present invention, adequate separation of the particle size distribution generated by QD self-assembly is achieved by, for example, modulation of the buffer and sample flow rates using automatic feedback control of electrokinetic driving voltages based on the real-time optical signals.

The present inventors have discovered that microfluidic design challenges are minimized through particle size distribution from QD self-assembly. Assessments of the self-assembled QD aggregate particle size distribution have been developed using dynamic light scattering instrumentation. These measurements can be made as a function of mixing time, QD:antibody ratio and QD:antigen ratio. The resulting data is suitable for mathematical representation to facilitate optimization relative to the capabilities of the microfluidic device. Biomarker-specific self-assembly results in QD aggregates from 200 nm to 2000 nm with fluorescence intensities at least 10-fold greater than unreacted QD, enabling discrimination of aggregates and unreacted QDs by fluorescence intensity.

Embodiments of the present invention include multiple QD colors, each presenting the antibody for a single, unique breast cancer biomarker. This approach includes compensation for nonspecific antibody-biomarker binding using methods developed for kinetic ELISA that distinguishes specific from nonspecific immunoassay interactions in the time domain based on differences in equilibrium binding rate coefficients.

Chips of the present invention may be fabricated with the appropriately surface functionalized QDs preloaded in reservoirs. The cellular components are removed from a drop of blood by flow focusing and the remaining plasma is combined with the QDs in the device. Controlled mixing is performed in on-chip reservoirs of special design to generate the self-assembled aggregates that are flow focused through optical detectors. Since QDs can typically be excited at a common wavelength (410 nm), the light from a single fiber coupled laser (Lasermate Group, Inc., CA, USA) is distributed to all five optical interrogation locations.

Detection of the fluorescence emission is carried out with optical filters corresponding to the four QD colors (MK Photonics, Albuquerque, N. Mex.) and a silicon photodiode array (Hamamatsu, USA). The photodiode array includes 10 Si-PIN photodetectors and each can be coupled with a 100 μm fiber. After electronic amplification, the collected signals are analyzed and stored on the hand-held device. The sensing (photo-detecting) fibers approach the channel and the particles from the bottom of the chip. The excitation light is introduced by optical fibers from the top of the chip. The fiber ends touch the bottom glass plate and the top PDMS plate and a fiber positioner will be designed to hold and align the fibers with the fluidic channel.

Device performance is assessed in buffer samples containing biomarker(s) for characterization of optimal performance.

### EXAMPLES

The following examples are presented to be exemplary of certain aspects of the present invention, and are not to be construed to be limiting thereof.

### Example 1

### Antigen Mediates Formation of Microscale Aggregates from Antibody-Coated QDs

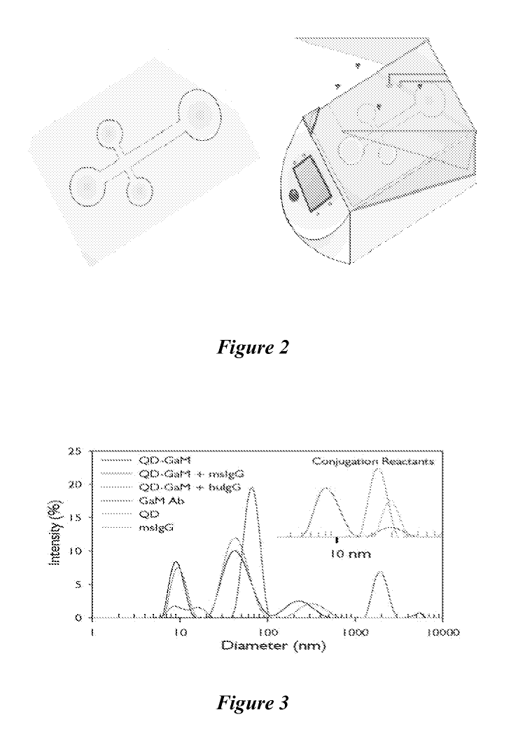

Functionalization of quantum dot streptavidin conjugate (QD; Invitrogen, Q10161MP) with biotin-conjugated goat anti-mouse IgG (GaM; BD Biosciences, 553999) was done as described in literature (Goldman, E. R., et al., Avidin: A Natural Bridge for Quantum Dot-Antibody Conjugates. Journal of the American Chemical Society, 2002. 124(6378-6382)) and verified by dynamic light scattering (FIG. 2). Nanoscale QD-GaM forms microscale aggregates mediated by the addition of the mouse IgG antigen (Mus; Polysciences, 23873). Addition of 4 μmol/L Mus mediates the appearance of a new particle population with diameters greater than 1,000 nm as detected by laser light scattering (FIG. 3). The formation of antigen-mediated QD aggregates greater than 700 nm in diameter is critical to the proposed approach and is documented for the first time here. The shift in mean particle diameter of the QD-GaM from 40 nm to 70 nm suggests the formation of antigen-decorated QD-GaMs that do not participate in the formation of large aggregates. Addition of 4 μmol/L human IgG antigen (Hum; Polysciences, 23872) as a nonspecific control fails to mediate the generation of particles greater than 700 nm diameter or modulate the other particle size distribution peaks in a significant way, confirming the immunospecificity of the assay (FIG. 2).

### Example 2

### The Subpopulation of QD-GaM-Mus Aggregates Identified by Flow Cytometry

QD-GaM interaction with antigen (1 or 100 μg/mL Mus; FIG. 4, panels B and C, respectively) or control (PBS buffer or 100 μg/mL Hum; FIG. 4, panels A and D, respectively) was carried out in a standard flow cytometry tube (1 mL total volume) for one hour at room temperature. Events were collected using Becton-Dickinson FACSCalibur with detection parameters optimized for relevant events. Fluorescence intensity in the FL3 channel (appropriate for QD705) was the primary event trigger. Quad regions were established in the SSC vs. FSC domain to isolate, and identify through color gating (red for Mus, blue for Hum), events with light scatter characteristics consistent with particle diameters greater than approximately 430 nm (FSC >10 arbitrary units (a.u.)). Specific protein detection sensitivity has been improved by 5 orders of magnitude (to 100 pg/mL) using the 405 nm excitation available on a BD Biosciences FACSAria flow cytometer. Optimized QD-GaM:msIgG stoichiometry was also used to achieve this improved detection sensitivity and involved a reduction in the relative QD-GaM concentration by 3 orders of magnitude. Specific agglomeration is 10-fold greater than non-specific agglomeration under the optimized conditions and for a msIgG concentration of 100 pg/mL.

### Example 3

### Electrokinetically Controlled Flow Focusing Separates Single Particles from a Particle Mixing Reservoir

Electrokinetic flow focusing can be achieved using a cross-shaped microchannel (FIG. 5). The ability to control the flow focusing in a cross microchannel has been demonstrated, as well as focusing the particle-carrying stream in a flow cytometer chip.

### Example 4

### Optical Sensing of a Particle Flowing in an Microchannel Flow

20 μm particles flowing in a microchannel under the influence of electrokinetic control (as in FIG. 5) are sufficiently separated to produced discrete optical triggering (FIG. 6). A small semiconductor laser and a Si-PIN detector are used for the optical detection. The optical detection system will be expanded in this work to include five detectors with the combined capability for counting particles, measuring particle velocity, identifying particle sizes and characterizing fluorescence intensity at four different emission wavelengths. Evidence particle counting in a flow cytometer chip using embedded optical fibers in the PDMS.

### Example 5

### Ang-2 Marker Detection

This example is similar to Example 1. Streptavidin-coated quantum dots with 705 nm (#Q10161MP), 585 nm (#Q10111MP), and 525 nm (#Q10141MP) emission wavelengths were purchased from Invitrogen (Carlsbad, Calif.) and used as received for flow cytometry, bulk agglomeration fluorescence and dynamic light scattering experiments respectively. Biotin conjugated anti-angiopoietin-2 polyclonal antibody (anti-ang-2) (#BAF623) and recombinant human angiopoietin-2 (ang-2) (#623-AN-025) were purchased from R&D Systems (Minneapolis, Minn.), reconstituted in Tris-buffered saline (TBS) containing 0.1% bovine serum albumin (BSA). Mouse IgG (mus) (#23873), human IgG (hum) (#23872) and rabbit IgG (rab) (#23874) were purchased from Polysciences Inc (Warrington, Pa.), and reconstituted in 1× phosphate buffered saline (PBS). All reconstituted samples were aliquoted and stored at −20° C. The aliquots were thawed and diluted to appropriate concentration using the PBS with 0.1% BSA (PBS-BSA) immediately prior to use. Biotin conjugated goat-anti-mouse IgG (GaM) (#553999) was purchased from BD Biosciences (San Jose, Calif.) and stored at 4° C. All other chemicals used were ACS reagent grade. 10 mM borate buffer was used for Zetasizer and bulk fluorescence measurement experiments. Buffers were prepared in deionized water, and filtered through a 0.2 μm filter prior to use.

Flow cytometric measurements were carried out on Beckton Dickinson (BD) FACSCalibur. BD FACSAria and BD LSR II flow cytometers were also used for optimizing detection parameters. Bulk fluorescence was measured in BioTek (Winooski, Vt.) Synergy HT multi-detection microplate reader. Dynamic light scatter (DLS) measurements were carried out on a Malvern Instruments (Malvern, UK) Zetasizer Nano ZS. Fluorescence measurements were carried out in a Nanodrop Technologies (Wilmington, Del.) ND-3300 fluorospectrometer.

The quantum dot-streptavidin conjugates (QD) and biotinylated anti-angiopoietin-2 polyclonal antibody (anti-ang-2) or biotinylated goat-anti-mouse polyclonal antibody (GaM) were mixed in PBS-BSA at QD:antibody molar ratio of 1:3 and 1 nM QD concentration. The conjugation was monitored by particle size estimation in the reaction mixture by DLS. The conjugate was diluted to appropriate concentrations and used immediately after synthesis.

The QD-antibody (QD-Ab) conjugate solution and the antigen or control solution at the appropriate dilutions and volumes were added to PBS-BSA for a total volume of 1 mL. BSA, similar to ang-2 in terms of molecular weight, also acted as a negative control for ang-2. Rab and hum were used as negative control for mus. The reaction mixtures were incubated at room temperature for 60 minutes and then analyzed by flow cytometry. Baseline event distribution of QD-Ab dispersed in PBS-BSA was also analyzed.

The candidate cancer biomarker protein, ang-2, was detected by flow cytometry to 0.5 pM concentration. Mus, which was used as a model protein in the initial experiments to optimize the instrument detection parameters and experimental conditions was also detected by flow cytometry to 0.5 pM concentration. The fraction of events classified as aggregates was 1.0+/−0.3%, compared to the negative control aggregate formation of 0.7+/−0.1%. Two different log-linear regimes were observed for aggregate formation, in a manner similar to that documented for ang-2. 10 pM QD-GaM was used to detect mus from 0.5 pM to 500 pM. 100 pM QD-GaM was used to detect mus from 500 pM to 500,000 pM. The slope of these relationships effectivly enabled resolution of [mus] between 0.5 pM and 500,000 pM.

### Example 6

### Agglomeration and Quantitative Detection

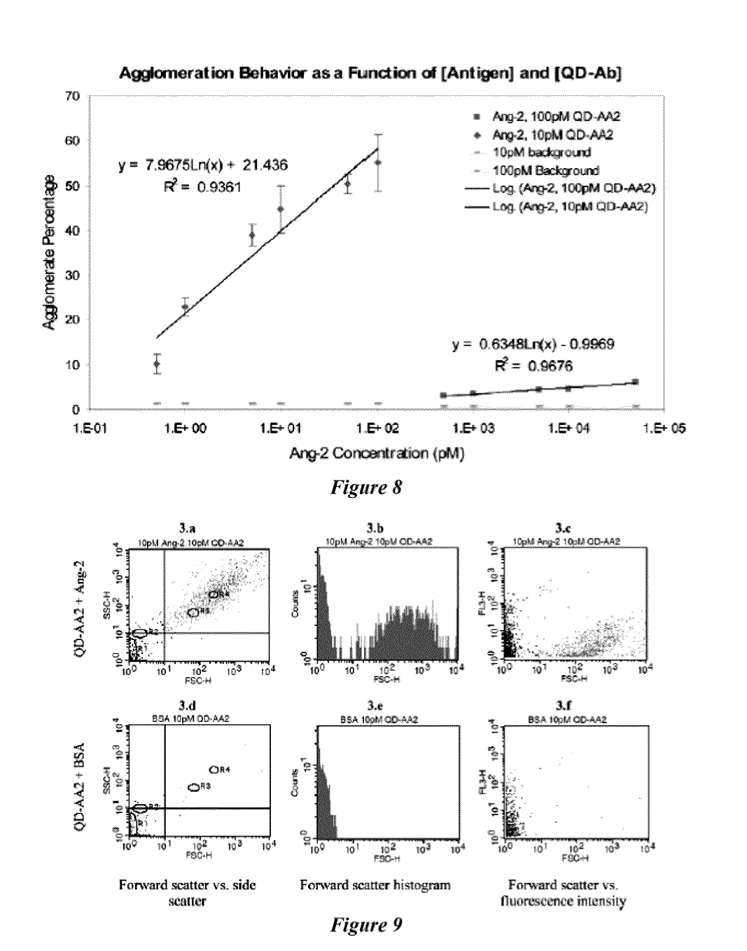

FIG. 8 shows that Ang-2 was detected down to 0.5 pM using the QD agglomeration technique. The percent of total events detected that were categorized as agglomerates (Y axis) is a log-linear function of the antigen concentration (X axis). Since the number of agglomerates in the two component reaction is limited by the availability of either or both of the components, the function is linear over a limited range. Hence, the agglomeration behavior of the lower concentration range of ang-2 (0.5 pM-100 pM) was linear when detected with 10 pM QD-AA2, while the higher concentration range of ang-2 (500 pM to 50000 pM) exhibited a log-linear agglomeration behavior with 100 pM QD-AA2. Data points are mean+/−standard deviation, n=3.

### Example 7

### Flow Cytometric Detection of Antigen

In this example, the inventors demonstrate an aspect of the present invention in which the percentage of self assembled agglomerates in a colloidal mixture can presumably be determined by flow cytometry using a variety of parametric combinations. We have utilized a combination of forward light scatter threshold and side light scatter threshold to demarcate agglomerates from smaller particles. The fraction of total events corresponding to the agglomerated sub-population serves as a metric correlated with antigen concentration. An example of the significant difference in the approximate size distribution of QD agglomerates mediated by ang-2 antigen in comparison with the BSA control appears as panels (FIG. 9) 9.b and 9.e, respectively. Forward light scatter intensity (FSC) is an approximate surrogate that is positively correlated with event diameter, suggesting that the addition of ang-2 mediates the formation of many aggregates significantly larger in diameter than can be triggered by the BSA control antigen. The correlation between forward light scatter and event size for this instrument is identified in panels 9.a and 9.d as the gated regions R1, R2, R3 and R4, which correspond to latex calibration sphere diameters of 0.2, 0.5, 1.0, and 2.0 μm, respectively. Events of these sizes are significantly larger than the diameter of antibody-functionalized QDs. Quadrant gating in the forward light scatter and side light scatter (SSC) space highlights events with diameters greater than approximately 0.5 μm (500 nm). The events in the upper right quadrant are highlighted in red and are defined to be QD agglomerates in this method. This gating also corresponds to the bimodal population distribution in the aggregated sample, as seen from the FSC histogram (panel 9.b). The addition of 10 pM ang-2 resulted in an agglomerate sub-population of 44% (panel 9.a), significantly greater than the 1.2% mediated by addition of the control BSA antigen (panel 9.d). The agglomerates identified by forward light scatter intensity are also fluorescent in the FL3 wavelength range (650 nm and longer), consistent with the fluorescence of QDs with an emission maxima of 705 nm (panels 9.c and 9.f).

The FL3-FSC representation (9.c, 9.f) provides an example of how the multiparametric data obtained from the flow cytometer enables sophisticated analysis of the sample, and may increase signal to noise ratio and sensitivity of detection. In this instance, two different populations of particles appear in the upper-right quadrant of the FSC-SSC space (9.a) but can not be distinguished from each other. However, in the forward scatter-fluorescence space (9.c), the non-specific agglomerates can be easily separated from the antigen mediated agglomerates. Most QD-AA2-ang2 agglomerates have high forward scatter and low fluorescence intensity (panel 9.c). While the volume of these agglomerates is about 250-fold greater than the individual QD-AA2 agglomerates, the fluorescence intensity is only 3-fold greater. A very small fraction of particles (less than 0.1%) in this agglomerated sample show high FSC as well as high FL3 intensities. These anomalous events are likely due to electronic noise as well as non-specific agglomeration between QD-AA2 conjugates. While these two populations appear in the same region on the FSC-SSC plot (9.a), they can be easily distinguished from each other in the FL3-FSC representation (9.c). In samples where a higher concentration of the QD-Ab conjugate is used, the number of these anomalous events is even larger. Combined with the smaller overall fraction of the agglomerate population in these samples, the increased utility of the multiparametric characterization to increase signal to noise ratios and detection sensitivity is apparent.

FIG. 9 shows this flow cytometric detection of antigen is achieved by characterizing agglomerates as a fraction of total events. In summary, each dot in panels a, d, c and f represents one particle or ‘event’ detected. Forward light scatter (FSC-H) and side light scatter (SSC-H) intensities are positively correlated with the size and complexity of the particles. In panels a and d, the ovals labeled R1 through R4 indicate standard latex beads of sizes 0.2, 0.5, 1.0, and 2.0 microns respectively, and provide an estimate of the diameter of the QD agglomerates detected. Panels b and e show the change in particle size distribution upon addition of the antigen. Panels c and f show the relation between fluorescence intensity (FL3) and size (FSC-H) for the agglomerates and the native QD-GaM respectively. The multivariate characterization of particles in the flow cytometer enables highly sophisticated analysis of the particles difficult to achieve by other methods including dynamic light scattering. This may increase the antigen detection sensitivity via better discrimination between specific and non specific self assembly. Detected values for the scatter and fluorescence intensities are digitized in1024 channels over the range of 1-10⁴ a.u. Typical data obtained from one experiment from n=3.

### Example 8

Materials: Streptavidin-coated quantum dots with 705 nm fluorescence emission (QD705, #Q10161MP) were purchased from Invitrogen (Carlsbad, Calif.) and used as received. Biotin conjugated anti-angiopoietin-2 polyclonal antibody (aA2, #BAF623), and recombinant human angiopoietin-2 (ang2, #623-AN) were purchased from R&D Systems (Minneapolis, Minn.), reconstituted in Tris-buffered saline (TBS) containing 0.1% bovine serum albumin (BSA) and stored at −20° C. Appropriate dilutions of all antibodies and antigens were prepared in phosphate buffered saline (PBS) with 0.1% BSA immediately prior to use. Deionized water with 18 MΩ·cm⁻¹ resistance was used for preparing buffers. All buffers were filtered through 0.2 μm filters. All other reagents were ACS reagent grade. Flow cytometric measurements were carried out with an unmodified Beckton Dickinson (BD) FACSCalibur, and the associated software.

Experimental: Quantum dot-streptavidin conjugates (QD) and biotinylated anti-angiopoietin-2 polyclonal antibody (aA2) were mixed in PBS-BSA at QD:antibody molar ratio of 1:3 and 1 nM QD concentration. The conjugate was incubated for 30 minutes at room temperature and then used as synthesized. The stoichiometry in this protocol has been previously optimized to obviate the need to separate un-reacted antibodies from the QD-Ab conjugates. The QD-aA2 conjugate solution and the ang2 or control solution at the appropriate dilutions and volumes were added to PBS-BSA for a final volume of 500 μL. BSA, similar to ang-2 in terms of molecular weight, was used as a negative control for Ang2. The reaction mixtures were incubated at room temperature and analyzed by flow cytometry at five minute intervals from t=5 minutes to t=90 minutes, as well as immediately after sample preparation, nominally t=1 minute. Thus, each experiment consisted of nineteen different samples, one for each time point. The influence of ang2 concentration on aggregation kinetics was characterized by carrying out the aggregation reaction with 1 pM, 10 pM, and 100 pM ang2. Control measurements were made at the same time points on samples identical except for the presence of ang2. To compensate for the effect of the variability in the QD-aA2 dilution on the fraction of agglomerates observed, only the datasets with base QD-aA2 count within 2000+/−200 were utilized for analysis. Three such datasets for each antigen concentration comprised the analyzed data set.

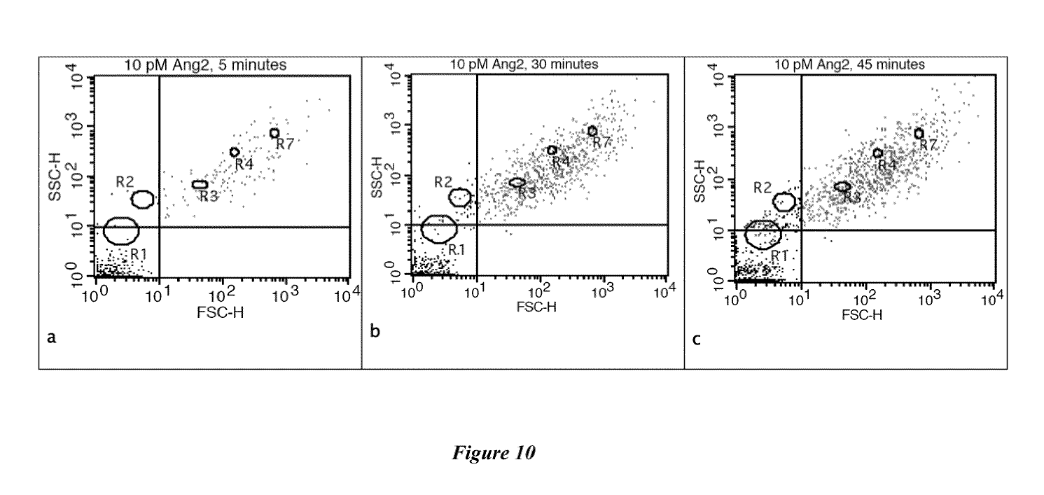

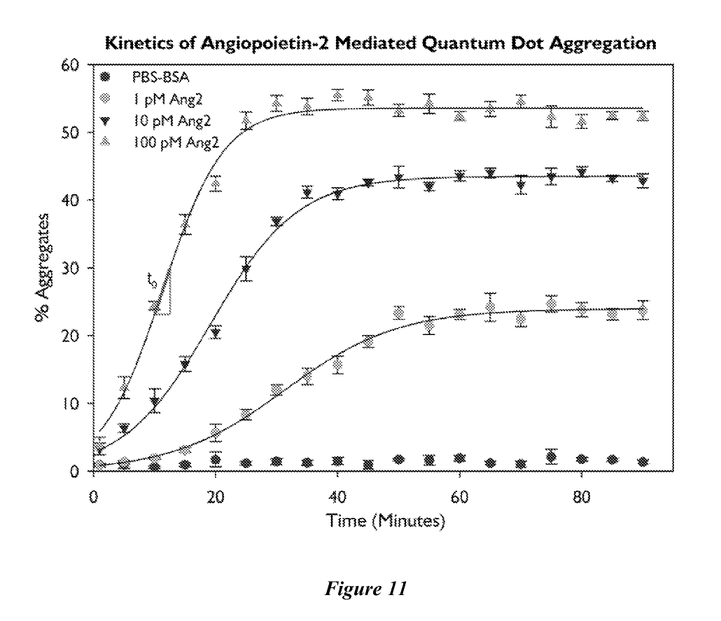

The size, fluorescence and number of the aggregates in the incubated samples were characterized by flow cytometry. The basic flow cytometric protocol followed was similar to the one used in our previous publication. Briefly, signal amplification parameters for the flow cytometer parameters forward light scatter (FSC) and side light scatter (SSC) detectors and fluorescence detector with 650 nm long pass filters (FL3) were optimized for characterizing small particles. The FSC and SSC detector performance was calibrated by using 0.2, 0.5, 1, 2 and 2.8 μm latex calibration particles. In FIG. 10, the regions indicated by R1, R2, R3, R4 and R7 correspond to the 0.2, 0.5, 1, 2 and 2.8 μm latex calibration particles respectively. The data was acquired at low flow rate (12+/−3 μL/min) for one minute. The FL3 channel was used as the event trigger consistent with the fluorescence emission characteristics of the 705 nm QDs used in this study. The fraction of aggregates was defined as the event fraction appearing in the upper right quadrant (UR) of the FSC vs. SSC plot. FSC greater than 10 a.u. corresponds to a nominal size greater than approximately 0.5 μm, as well as the observed differentiation of the individual QD-Ab conjugates from the large aggregates inherent in the agglomeration process and the measurement protocol. This fraction was calculated automatically by the CellQuest Pro software. Analysis of the datasets was carried out using the SigmaPlot 9.01 and SigmaStat 3.1 software.

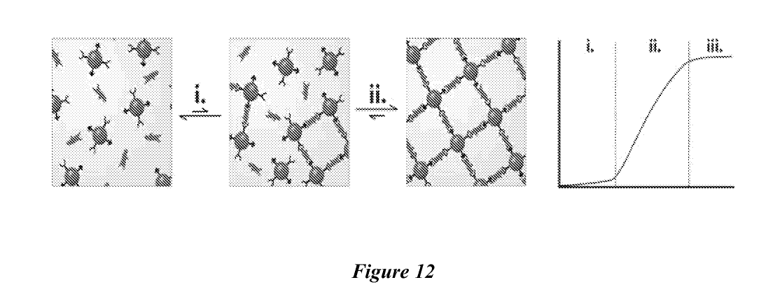

Results and Discussion: The kinetics of ang2 mediated QD-aA2 aggregation exhibits a sigmoidal behavior, with three distinct phases. The initial rate of aggregate formation is slow, most evident from the kinetics for aggregation triggered by the addition of 1 pM ang2 (FIG. 11). The second phase is characterized by rapid, self-assembled aggregation. The final phase is the reduction of aggregation rate with an asymptotic approach to the equilibrium aggregate fraction. Similar sigmoidal kinetics are observed in many intermolecular interaction based self-assembly processes as well for nanoparticle synthesis and polymer synthesis. Sigmoidal kinetics indicate a thermodynamically unfavorable intermediate in the reaction pathway. The key stages in the aggregation reaction are depicted schematically in FIG. 12 where the first panel represents the QD-Ab conjugates mixed with antigen molecules. Antibody-antigen recognition creates single QD-Ab:antigen complexes, which interact with each other, free QD-Ab conjugates and additional free antigen molecules. This is phase i of interactions which leads to the gradual formation of small complexes that act as nuclei for microscale aggregates. (FIG. 12, second panel). During the formation of multi-particle aggregates by molecular recognition, aggregation is favored by the decrease in free energy of the interacting particles, but the entropic cost associated with increased particle organization hinders nucleus formation. Consequently, the probability of formation of micron scale aggregates as well as the aggregation rate are initially low. Once the nuclei have reached the critical size, the presence of multiple binding sites on the nuclei results in rapid growth of the aggregate. This corresponds to phase ii, exhibiting rapid growth in the aggregate fraction. The transition from critical nucleus to large aggregates may occur rapidly. Flow cytometric characterization and dynamic light scattering data published previously, show only two particle populations—the individual, unaggregated QD-aA2:ang2 conjugates and the micron scale aggregates. While the fraction of micron-scale aggregates changes over time, an intermediate population is not apparent in these data. The lack of detectable concentrations of intermediate is consistent with the data published in the literature for other aggregation systems cited previously. Finally, as the individual components are depleted (FIG. 12, third panel), the aggregation rate slows and the system reaches a stable steady state.

Aggregation kinetics observed in the QD-aA2:ang2 system may be described by a three parameter, sigmoidal curve of the form

$$a = \frac{{a}_{max}}{1 - {e}^{- ( \frac{t - {t}_{0}}{τ} )}} \qquad ( Equation 1 )$$

In Equation 1, a and t are the variables representing aggregate fraction and time, respectively. The constants for the sigmoids are aₘₐₓ, t₀ and τ, where aₘₐₓ is the equilibrium value for a, t₀ is the time point of inflection, and τ is a time constant. These parameters are modulated by the ang2 concentration in contact with the biofunctionalized QDs (Table 1, below). The aₘₐₓ values obtained from the sigmoidal curves fit to the 1 pM, 10 pM, and 100 pM ang2 datasets are statistically equivalent to the steady state aggregate fraction observed previously for the respective ang2 concentrations. t₀, which describes the inflection point for the sigmoidal curve, decreases with increasing ang2 concentration. Nucleation-limited aggregation processes, such as the self-assembly described here, accelerate in response to increased concentration of the bridging agent. This behavior, consistently observed in other systems and predicted in many model representations is confirmed in the present work. Increased ang2 concentration results in more rapid achievement of micron sized aggregates from nanoscale QDs, consistent with an increased rate of interactions between the QD-aA2 conjugates and the free ang2 molecules in solution. Thus, increasing ang2 concentration causes earlier achievement of a significant nucleation subpopulation and more rapid transition to the aggregate growth phase. The effect of increasing ang2 concentration on aggregation rate is also evident in the difference in slopes of the aggregation curves. Antigen concentration modulates the rate of aggregate formation at the early time points during the low aggregation rate phase as well as at the inflection point (Table 1). The different slopes observed for different ang2 concentration indicate that an unknown concentration of the target antigen may be estimated from the rate of increase in aggregate count at incubation times far shorter than those required to achieve steady state. The optimal time point for discriminating among antigen concentrations may be determined through comparative analysis of aggregation profiles. The time constant τ also decreases with increasing antigen concentration and corresponds with the higher rate of aggregation during phase two following nucleation. The ang2 concentration is correlated with the sigmoid coefficients aₘₐₓ, t₀ and t, and the slopes at t=0 minutes and t=t₀, with log linear functions with correlation coefficients 0.96, 0.98, 0.99, 0.95 and 0.99 respectively. These correlation coefficient values further support the feasibility of quantitatively detecting the biomarker concentration from one or more of the aggregation kinetics parameters. The control samples in the absence of ang2, do not exhibit time dependent aggregation behavior (FIG. 11), further suggesting the potential to use kinetic assessments to detect specific antigens in the presence of other proteins.

TABLE 1
Table 1: Parameters of the Aggregation Kinetics Sigmoid are Modulated by Antigen Concentration.

| Angiopoietin2 | | | | | |
|---|---|---|---|---|---|
| concentration | aₘₐₓ | t₀ | τ | slope, t = 5 min | slope, t = t₀ |
| (picomolar) | (percent) | (minutes) | (minutes) | (percent/minute) | (percent/minute) |
| 1 | 23.98 +/− 0.35 | 31.41 +/− 0.38 | 9.27 +/− 0.76 | 0.13 +/− 0.02 | 0.65 +/− 0.05 |
| 10 | 43.51 +/− 0.35 | 19.18 +/− 0.84 | 7.12 +/− 0.16 | 0.65 +/− 0.05 | 1.52 +/− 0.05 |
| 100 | 53.57 +/− 0.30 | 11.35 +/− 0.53 | 4.96 +/− 0.21 | 1.83 +/− 0.09 | 2.68 +/− 0.11 |

Angiopoietin-2 concentration has quantifiable effects on QD-aA2 aggregation kinetics. The sigmoid curves fit to the aggregation data are described by three constants aₘₐₓ, t₀ and τ. The equilibrium aggregate percentage, aₘₐₓ, increases with increasing ang2 concentration, in agreement with previously published data. The inflection time t₀, and time constant τ both decrease with increasing ang2 concentration, indicating faster nucleation and growth of aggregates as a result of higher ang2 concentration. The rates of aggregation, represented by slopes of the sigmoidal curves are also a function of ang2 concentration, including at t=5 minutes, and at t=t₀. The concentration dependent aggregation rate may enable rapid detection and quantification of target antigens, as well as resolution of specific and no-specific intermolecular interaction. The values represent mean sigmoid coefficients and slopes obtained by fitting a three parameter sigmoid function to three datasets individually, and the respective standard deviations. The slopes at t=5 minutes and t=t₀ were obtained by fitting a linear function to eight sigmoid curve data points centered on the corresponding time points, as generated by Sigmaplot.

The following references, incorporated herein by reference in their entirety, are related to the application in general and more specifically Example 8.
1. Zlotnick, A. and Stray, S. J., How does your virus grow? Understanding and interfering with virus assembly. Trends in Biotechnology, 2003. 21(12): 536-542.
2. Flyvbjerg, H.; Jobs, E. and Leibler, S., Kinetics of self-assembling microtubules: an “inverse problem” in biochemistry. Proceedings of the National Academy of Sciences of the United States of America, 1996. 93(12): 5975-5979.
3. Na, G. C.; Butz, L. J. and Carroll, R. J., Mechanism of in vitro collagen fibril assembly. Kinetic and morphological studies. Journal of Biological Chemistry, 1986. 261(26): 12290-12299.
4. Whitesides, G. M.; Mathias, J. P. and Seto, C. T., Molecular self-assembly and nanochemistry: a chemical strategy for the synthesis of nanostructures. Science, 1991. 254(5036): 1312-1319.
5. Yan, H.; Park, S. H.; Finkelstein, G.; Reif, J. H. and LaBean, T. H., DNA-Templated Self-Assembly of Protein Arrays and Highly Conductive Nanowires. 2003, American Association for the Advancement of Science. p. 1882-1884.
6. Storhoff, J. J.; Elghanian, R.; Mucic, R. C.; Mirkin, C. A. and Letsinger, R. L., One-pot colorimetric differentiation of polynucleotides with single base imperfections using gold nanoparticle probes. J. Am. Chem. Soc, 1998. 120(9): 1959-1964.
7. Hirsch, L. R.; Jackson, J. B.; Lee, A.; Halas, N. J. and West, J. L., A Whole Blood Immunoassay Using Gold Nanoshells. Analytical Chemistry, 2003. 75: 2377-2381.
8. Soman, C. P. and Giorgio, T. D., Quantum Dot Self-Assembly for Protein Detection with Sub-Picomolar Sensitivity. Langmuir, 2008. 24(8): 4399-4404.
9. Goldman, E. R.; Balighian, E. D.; Mattoussi, H.; Kuno, M. K.; Mauro, J. M.; Tran, P. T. and Anderson, G. P., Avidin: a natural bridge for quantum dot-antibody conjugates. J. Am. Chem. Soc, 2002. 124(22): 6378-6382.
10. Zlotnick, A.; Aldrich, R.; Johnson, J. M.; Ceres, P. and Young, M. J., Mechanism of Capsid Assembly for an Icosahedral Plant Virus. Virology, 2000. 277(2): 450-456.
11. Flyvbjerg, H. and Jobs, E., Microtubule dynamics. II. Kinetics of self-assembly. Physical Review E, 1997. 56(6): 7083-7099.
12. Bonfils, C.; Bec, N.; Lacroix, B.; Harricane, M. C. and Larroque, C., Kinetic Analysis of Tubulin Assembly in the Presence of the Microtubule-associated Protein TOGp. Journal of Biological Chemistry, 2007. 282(8): 5570.
13. Koo, B. W. and Miranker, A. D., Contribution of the intrinsic disulfide to the assembly mechanism of islet amyloid. Protein Science, 2005. 14(1): 231.
14. Sluzky, V.; Tamada, J. A.; Klibanov, A. M. and Langer, R., Kinetics of insulin aggregation in aqueous solutions upon agitation in the presence of hydrophobic surfaces. Proceedings of the National Academy of Sciences of the United States of America, 1991. 88(21): 9377-9381.
15. Speed, M. A.; King, J. and Wang, D. I. C., Polymerization Mechanism of Polypeptide Chain Aggregation. BIOTECHNOLOGY AND BIOENGINEERING, 1997. 54(4).
16. Spirito, M. D.; Chiappini, R.; Bassi, F. A.; Stasio, E. D.; Giardina, B. and Arcovito, G., Aggregation kinetics and structure of cryoimmunoglobulins clusters. Physica A: Statistical Mechanics and its Applications, 2002. 304(1-2): 211-219.
17. Endres, D.; Miyahara, M.; Moisant, P. and Zlotnick, A., A reaction landscape identifies the intermediates critical for self-assembly of virus capsids and other polyhedral structures. Protein Science, 2005. 14(6): 1518.
18. Endres, D. and Zlotnick, A., Model-Based Analysis of Assembly Kinetics for Virus Capsids or Other Spherical Polymers. Biophysical Journal, 2002. 83(2): 1217-1230.
19. Hagan, M. F. and Chandler, D., Dynamic Pathways for Viral Capsid Assembly. Biophysical Journal, 2006. 91(1): 42.
20. McPherson, A., Micelle formation and crystallization as paradigms for virus assembly. BioEssays, 2005. 27(4): 447-458.
21. Zlotnick, A., Theoretical aspects of virus capsid assembly. J. Mol. Recognit, 2005. 18: 479-490.
22. Zlotnick, A.; Johnson, J. M.; Wingfield, P. W.; Stahl, S. J. and Endres, D., A theoretical model successfully identifies features of hepatitis B virus capsid assembly. Biochemistry, 1999. 38(44): 14644-14652.
23. Bray, D. and Lay, S., Rapid numerical integration algorithm for finding the equilibrium state of a system of coupled binding reactions. Bioinformatics, 1994. 10(5): 471-476.
24. Bray, D. and Lay, S., Computer-based analysis of the binding steps in protein complex formation. Proceedings of the National Academy of Sciences, 1997. 94(25): 13493.
25. Lay, S. and Bray, D., A computer program for the analysis of protein complex formation. Bioinformatics, 1997. 13(4): 439-444.
26. Chong, C. R. and Sullivan, D. J., Inhibition of heme crystal growth by antimalarials and other compounds: implications for drug discovery. Biochemical Pharmacology, 2003. 66(11): 2201-2212.
27. Kodaka, M., Interpretation of concentration-dependence in aggregation kinetics. Biophysical Chemistry, 2004. 109(2): 325-332.
28. Hancock, K. and Tsang, V. C., Development and optimization of the FAST-ELISA for detecting antibodies to Schistosoma mansoni. J Immunol Methods, 1986. 92(2): 167-76.
29. Kodaka, M., Requirements for generating sigmoidal time-course aggregation in nucleation-dependent polymerization model. Biophysical Chemistry, 2004. 107(3): 243-253.
30. Towey, T. F.; Khan-Lodhi, A. and Robinson, B. H., Kinetics and mechanism of formation of quantum-sized cadmium sulphide particles in water-aerosol-OT-oil microemulsions. Journal of the Chemical Society, Faraday Transactions, 1990. 86(22): 3757-3762.
31. Matsumoto, A.; Kodama, K.; Aota, H. and Capek, I., Kinetics of emulsion crosslinking polymerization and copolymerization of allyl methacrylate. European Polymer Journal, 1999. 35(8): 1509-1517.
32. Sabaté, R. and Gallardo, M., An Autocatalytic Reaction as a Model for the Kinetics of the Aggregation of-Amyloid. Biopolymers (Peptide Science), 2003. 71: 190-195.
33. Jarrett, J. T. and Lansbury Jr, P. T., Seeding “one-dimensional crystallization” of amyloid: a pathogenic mechanism in Alzheimer's disease and scrapie? Cell, 1993. 73(6): 1055-8.

Throughout this application, and specifically, below, various references are mentioned. All references are incorporated herein by reference in their entirety and should be considered to be part of this application.
1. L. L. Humphrey, et al., Breast Cancer Screening: A Summary of the Evidence for the U.S. Preventive Services Task Force. Annals of Internal Medicine, 2002. 137(5): p. 347-360.
2. Jemal, A., et al., Cancer statistics, 2004. CA Cancer J Clin, 2004. 54(1): p. 8-29.
3. Calvo, K. R., L. A. Liotta, and E. F. Petricoin, Clinical Proteomics: From Biomarker Discovery and Cell Signaling Profiles to Individualized Personal Therapy. Bioscience Reports, 2005. 25(1/2): p. 107-125.
4. Meyerson, M. and D. Carbone, Genomic and Proteomic Profiling of Lung Cancers: Lung Cancer Classification in the Age of Targeted Therapy. Journal of Clinical Oncology, 2005. 23(14): p. 3219-3226.
5. Petricoin, E. F. and L. A. Liotta, Proteomic approaches in cancer risk and response assessment. Trends in Molecular Medicine, 2004. 10(2): p. 59-64.
6. Petricoin, E. F. and L. A. Liotta, SELDI-TOF-based serum proteomic pattern diagnostics for early detection of cancer. Current opinion in biotechnology, 2004. 15(1): p. 24-30.
7. Roboz, J., Mass spectrometry in diagnostic oncoproteomics. Cancer investigation, 2005. 23(5): p. 465-78.
8. Yanagisawa, K., et al., Proteomic patterns of tumour subsets in non-small-cell lung cancer. Lancet, 2003. 362(9382): p. 433-9.
9. Yanagisawa, K., et al., Molecular fingerprinting in human lung cancer. Clin Lung Cancer, 2003. 5(2): p. 113-8.
10. Plebani, M., Proteomics: The next revolution in laboratory medicine? Clinica Chimica Acta, 2005. 357: p. 113-122.
11. Righettia, P. G., et al., Proteome analysis in the clinical chemistry laboratory: Myth or reality? Clinica Chimica Acta, 2005. 357 p. 123-139.
12. Westermeier, R. and R. Marouga, Protein Detection Methods in Proteomics Research. Bioscience Reports, 2005. 25(1/2): p. 19-32.
13. Lupu, R., R. B. Dickson, and M. E. Lippman, The role of erbB-2 and its ligands in growth control of malignant breast epithelium. J Steroid Biochem Mol Biol, 1992. 43(1-3): p. 229-236.
14. Wu, J. T., C-erbB2 oncoprotein and its soluble ectodomain: a new potential tumor marker for prognosis early detection and monitoring patients undergoing Herceptin treatment. Clin Chim Acta, 2002. 322(1-2): p. 11-19.
15. Eccles, S. A., The role of c-erbB-2/HER2/neu in breast cancer progression and metastasis. J Mammary Gland Biol Neoplasia, 2001. 6(4): p. 393-406.
16. Imoto, S., T. Kitoh, and T. Hasebe, Serum c-erB-2 levels in monitoring of operable breast cancer patients. Jpn J Clin Oncol, 1999. 29(7): p. 336-9.
17. Hudelist, G., et al., Serum EGFR levels and efficacy of trastuzumab-based therapy in patients with metastatic breast cancer. Eur J Cancer, 2006. 42(2): p. 186-92.
18. Lafky, J. M., et al., Serum soluble epidermal growth factor receptor concentrations decrease in postmenopausal metastatic breast cancer patients treated with letrozole. Cancer Res, 2005. 65(8): p. 3059-62.
19. Muller, V., et al., Prognostic and predictive impact of soluble epidermal growth factor receptor (sEGFR) protein in the serum of patients treated with chemotherapy for metastatic breast cancer. Anticancer Res, 2006. 26(2B): p. 1479-87.
20. Huflejt, M. E. and H. Leffler, Galectin-4 in normal tissues and cancer. Glycoconj J, 2004. 20(4): p. 247-55.
21. Iurisci, I., et al., Concentrations of galectin-3 in the sera of normal controls and cancer patients. Clin Cancer Res, 2000. 6(4): p. 1389-93.
22. Moiseeva, E. V., et al., Galectins as markers of aggressiveness of mouse mammary carcinoma: towards a lectin target therapy of human breast cancer. Breast Cancer Res Treat, 2005. 91(3): p. 227-41.
23. Zou, J., et al., Peptides specific to the galectin-3 carbohydrate recognition domain inhibit metastasis-associated cancer cell adhesion. Carcinogenesis, 2005. 26(2): p. 309-18.
24. Kossoy, G., et al., Human soluble p66 and p51 tumor-associated antigens promote the suppression of rat mammary tumors in comparison to commercial human albumin. Oncol Rep, 2004. 11(2): p. 487-91.
25. Goldman, E. R., et al., Avidin: A Natural Bridge for Quantum Dot-Antibody Conjugates. Journal of the American Chemical Society, 2002. 124(6378-6382).
26. Ren, L. and D. Li, Theoretical studies of microfluidic dispensing processes. J Colloid Interface Sci., 2002. 254: p. 384-395.
27. Ren, L., D. Sinton, and L. D., Numerical simulation of microfluidic injection processes in crossing microchannels. Journal of Micromechanics and Microengineering, 2003: p. 739-47.
28. Sinton, D., L. Ren, and D. Li, A dynamic loading method for controlling on-chip microfluidic sample injection. J Colloid Interface Sci., 2003. 266: p. 448-456.
29. Sinton, D., L. Ren, and D. Li, Visualization and numerical modelling of microfluidic on-chip injection processes. J Colloid Interface Sci., 2003. 260: p. 431-439.
30. Sinton, D., et al., Effects of liquid conductivity differences on multi-component sample injection, pumping and stacking in microfluidic chips. Lab Chip, 2003. 3: p. 173-79.
31. Xuan, X. and D. Li, Focused electrophoretic motion and selected electrokinetic dispensing of particles and cells in cross-microchannels. Electrophoresis, 2005. 26: p. 3552-3560.
32. Xiang, Q., et al., Multi-functional Particle Detection with Embedded Optical Fibers in a Poly(dimethylsiloxane) Chip. Instrumentation Sci & Tech., 2005. 33: p. 597-607.
33. Barlough, J. E., et al., The kinetics-based enzyme-linked immunosorbent assay for coronavirus antibodies in cats: calibration to the indirect immunofluorescence assay and computerized standardization of results through normalization to control values. Can J Vet Res, 1987. 51(1): p. 56-9.
34. Barlough, J. E., et al., Evaluation of a computer-assisted, kinetics-based enzyme-linked immunosorbent assay for detection of coronavirus antibodies in cats. J Clin Microbiol, 1983. 17(2): p. 202-17.
35. Barlough, J. E., et al., Coronavirus antibody detection in cats by computer-assisted kinetics-based enzyme-linked immunosorbent assay (KELA): field studies. Cornell Vet, 1986. 76(3): p. 227-35.
36. Hancock, K. and V. C. Tsang, Development and optimization of the FAST-ELISA for detecting antibodies to Schistosoma mansoni. J Immunol Methods, 1986. 92(2): p. 167-76.
37. Spitalnik, S., et al., A new technique in quantitative immunohematology: solid-phase kinetic enzyme-linked immunosorbent assay. Vox Sang, 1983. 45(6): p. 440-8.
38. Tsang, V. C., B. C. Wilson, and S. E. Maddison, Kinetic studies of a quantitative single-tube enzyme-linked immunosorbent assay. Clin Chem, 1980. 26(9): p. 1255-60.

Various changes in the details, steps and materials that have been described may be made by those skilled in the art within the principles and scope of the invention herein illustrated and defined in the appended claims. Therefore, while the present invention has been shown and described herein in what is believed to be the most practical and preferred embodiment, it is recognized that departures can be made therefrom within the scope of the invention, which is therefore not to be limited to the details disclosed herein but is to be accorded the full scope of the claims so as to embrace any and all equivalent apparatus and methods.

Unless otherwise indicated, all numbers expressing quantities, specifically amounts set forth when describing experimental testing, are to be understood as being modified in all instances by the term “about.” Accordingly, unless indicated to the contrary, the numerical parameters set forth herein are approximations that may vary depending upon the desired properties sought to be determined by the present invention.

We claim:

1. A method of detecting a biomarker, comprising:
coupling quantum dots to a molecular recognition element that binds to a biomarker of interest to form functionalized conjugates;
providing a sample from a subject;
introducing the sample and the functionalized conjugates to at least one sample holding channel in an incubation chamber to form agglomerates through self-assembly if contacted with the corresponding biomarker;
detecting the agglomerate by at least one of the rate of agglomeration and/or the number of agglomerates.

2. The method of claim 1, wherein the detecting step includes correlating antigen concentration with agglomerate formation.

3. The method of claim 1, wherein the molecular recognition element is an antibody.

4. The method of claim 1, wherein the rate of agglomeration correlates with a disease state.

5. The method of claim 4, wherein the disease state is cancer.

6. The method of claim 1, comprising coupling a second set of quantum dots to a second molecular recognition element that binds to a second biomarker of interest to form a second set of functionalized conjugates;
introducing the sample and the second set of functionalized conjugates to at least one sample holding channel in an incubation chamber to form a second set of agglomerates through self-assembly if contacted with the corresponding biomarker; and
detecting the second set of agglomerates by at least one of the rate of agglomeration and/or the number of agglomerates.

7. The method of claim 1, wherein the biomarker is indicative of a cancer.

8. The method of claim 1, wherein the biomarker is indicative of cancer metastasis.

9. The method of claim 7, wherein the cancer is one that metastasizes in bone.

10. The method of claim 9, wherein the cancer is lung, breast, or prostate cancer.

11. The method of claim 1, wherein the biomarkers include at least one of a protein, protein fragment, DNA, RNA, or oligosaccharide.

12. The method of claim 1, wherein the biomarker is at least one of TGF-β1, osteoprotegerin, parathyroid hormone protein, and bone specific alkaline phosphatase.

13. The method of claim 1, wherein the biomarker is at least one of c-ErB-2, sEGFR, or galectin-3.

14. The method of claim 1, wherein self-assembly results in at least a 1000-fold or greater change in volume when compared to a single quantum dot.
